(19) United States
(12) Patent Application Publication
(10) Pub. No.: US 2024/0226432 A1
(43) Pub. Date: Jul. 11, 2024

(54) ADD-ON DEVICE FOR BLOOD SET HANDPUMP WITH MECHANICAL LEVERAGE

(71) Applicant: CareFusion 303, Inc, San Diego, CA (US)

(72) Inventors: Shreya B. JOGLEKAR, Huballi (IN); Abin AUSTIN, Thrissur (IN); Deepak UDUPA, Mangalore (IN); Kaushik SUMAN, Jharkhand (IN); Sumit RAJPAL, Panipat (IN); Amarsinh Deeliprao JADHAV, Hyderabad (IN); NarsiReddy SANIKOMMU, Kanigiri (IN); Shashwat JAIN, Indore (IN); Bhanupratapsingh Dharmendrasingh THAKORE, Gujarat (IN); Anil Basavaraj NADAGOUDA, Bangalore (IN); Aman DESAI, Bengaluru (IN); Abhishek KRISHNAN, Bengaluru (IN)

(21) Appl. No.: 18/407,973

(22) Filed: Jan. 9, 2024

Related U.S. Application Data
(60) Provisional application No. 63/438,481, filed on Jan. 11, 2023.

(51) Int. Cl. A61M 5/168

(52) U.S. Cl. CPC A61M 5/16877; A61M 2205/3334

(57) ABSTRACT

Described herein is an add-on device for an intravenous (IV) set hand pump comprising a first member configured to actuate the handpump, a second member hingedly connected to the first member and configured to receive the hand pump, wherein, when actuated, the add-on device is configured to increase a fluid flow rate downstream of the IV set hand pump beyond a maximum gravity-based fluid flow rate of the IV set.

### CROSS REFERENCE TO RELATED APPLICATION

This application claims the benefit of priority under 35 U.S.C. § 119 to U.S. Provisional Patent Application Ser. No. 63/438,481, entitled “ADD-ON DEVICE FOR BLOOD SET HANDPUMP WITH MECHANICAL LEVERAGE”, filed on Jan. 11, 2023, the entire contents of which is incorporated herein by reference.

### FIELD OF THE INVENTION

The present disclosure generally relates to medical fluid distribution systems, and, in particular, to an add-on device to provide mechanical leverage for a hand pump for distributing a medical fluid from an IV bag.

### BACKGROUND

Medical treatments often include the infusion of a medical fluid (e.g., a saline solution or a liquid medication) to patients using an infusion pump that distributes the medical fluid from an intravenous (IV) bag to a patient. When an infusion pump is unavailable, medical practitioners use their hands to compress or otherwise squeeze on the IV bag to administer the medical fluid.

### SUMMARY

Manually compressing the IV bag can result in hand fatigue which greatly inconveniences medical practitioners. As such, there is a need for devices and methods of use thereof that can distribute medical fluids from an intravenous (IV) bag to a patient without fatiguing a medical practitioner. The disclosed subject matter relates to an add-on device to provide mechanical leverage for a hand pump for distributing a medical fluid from an IV bag. In certain implementations, the add-on device is a standalone reusable device with a hinged design consisting of two parts. The hand pump is placed on the second member and the first member actuates the pump to increase a flow rate of fluid in the IV set while lessening the hand fatigue of the medical practitioner.

Embodiments of the present disclosure provide an add-on device for an intravenous (IV) set hand pump comprising a first member configured to actuate the handpump, a second member hingedly connected to the first member and configured to receive the hand pump, wherein,

when actuated, the add-on device is configured to increase a fluid flow rate downstream of the IV set hand pump beyond a maximum gravity-based fluid flow rate of the IV set. In some embodiments, the first member comprises a compression section that extends into the hand pump when the first member is compressed towards the second member. In some embodiments, the compression section includes a portion of the first member that gradually compresses the hand pump by compressing a portion of the hand pump closest to the rotational axis and continues to compress sequential portions of the hand pump as the first member continues to rotate toward the second member. In some embodiments, the compression section includes arcuate portions that have varying degrees of curvature along the first member such that the hand pump is compressed in a rolling fashion from one end of the hand pump toward an opposite end of the hand pump as the first member is compressed towards the second member.

In some embodiments, the second member further comprises a ledge configured to secure the hand pump in the add-on device. In some embodiments, the first member and/or the second member comprises a grip portion. In some embodiments, the grip portion comprises a shaped surface having one or more grip members comprising at least one of a circular shape, an oblong shape and an outer arcuate groove. In some embodiments, each grip member is sized and shaped to receive a finger of a hand. In some embodiments, the distance between the first member and the second member is not fixed.

In some embodiments, the device is reusable by disconnecting with a first IV set and attaching to a second IV set without disconnecting either the first or second IV sets from a fluid source or from a delivery catheter assembly. In some embodiments, the device provides a mechanical leverage of three. In some embodiments, the first member and/or the second member further comprises a window for viewing the hand pump. In some embodiments, the overall length of the add on device is 225 mm, 230 mm, 235, mm, 240 mm, 245 mm, 250 mm, 255 mm, 260 mm, 265 mm, or 270 mm.

Embodiments of the present disclosure provide a method for using an add-on device for an intravenous (IV) set hand pump comprising coupling a first IV tube of an IV set to a fluid source, coupling a second IV tube of the IV set to a fluid recipient, installing the device to the IV set hand pump coupled between the first IV tube and the second IV tube, compressing the add-on device to force fluid to flow through the IV set hand pump at a flow rate greater than one of a maximum gravity-based fluid flow rate of the IV set and a maximum infusion flow rate of an infusion pump, and repeating the compressing and expanding steps to deliver a desired volume of fluid to the fluid recipient.

In some embodiments, the add-on device comprises a first member configured to actuate the handpump, a second member hingedly connected to the first member and configured to receive the hand pump, wherein, when actuated, the add-on device is configured to increase a fluid flow rate downstream of the IV set hand pump beyond a maximum gravity-based fluid flow rate of the IV set. In some embodiments, the first member and/or the second member comprises a grip portion. In some embodiments, the grip portion comprises a shaped surface having one or more grip members comprising at least one of a circular shape, an oblong shape and an outer arcuate groove, wherein each grip member is sized and shaped to receive a finger of a hand.

In some embodiments, the method further comprises reusing the device by disconnecting with a first IV set and attaching to a second IV set without disconnecting either the first or second IV sets from a fluid source or from a delivery catheter assembly.

Embodiments of the present disclosure provide a method for increasing fluid flow rate downstream of an IV set hand pump beyond a maximum gravity-based fluid flow rate of the IV set comprising coupling a first IV tube of the IV set to a fluid source, coupling a second IV tube of the IV set to a fluid recipient, installing an add-on device to the IV set hand pump coupled between the first IV tube and the second IV tube, and compressing the add-on device to force fluid to flow through the IV set hand pump.

In some embodiments, the add-on device comprises a first member configured to actuate the handpump, a second member hingedly connected to the first member and configured to receive the hand pump, wherein, when actuated, the add-on device is configured to increase a fluid flow rate downstream of the IV set hand pump beyond a maximum gravity-based fluid flow rate of the IV set.

The foregoing and other features, aspects and advantages of the disclosed embodiments will become more apparent from the following detailed description and accompanying drawings.

Note that the various implementations described above can be combined with other implementations described herein (e.g., keep an impedance for a single or group of neuromuscular-signal sensors can be combined with matching of impedances, such that impedances can be both matched and be kept within particular ranges of impedance values). The features and advantages described in the specification are not all inclusive and, in particular, additional features and advantages will be apparent to one of ordinary skill in the art in view of the drawings, specification, and claims. Moreover, it should be noted that the language used in the specification has been principally selected for readability and instructional purposes.

### BRIEF DESCRIPTION OF THE DRAWINGS

So that the present disclosure can be understood in greater detail, a more particular description may be had by reference to the features of various implementations, some of which are illustrated in the appended drawings. The appended drawings, however, merely illustrate pertinent features of the present disclosure. The description may admit to other effective features as the person of skill in this art will appreciate upon reading this disclosure.

FIG. 1A depicts a perspective view of an IV hand pump connecting a fluid source to a patient, according to aspects of the disclosure.

FIG. 1B depicts a perspective view of an example patient care system having four fluid infusion pumps, each of which is connected to a respective fluid supply for pumping the contents of the fluid supply to a patient.

FIG. 2 illustrates a conventional IV set showing the set connected to two fluid sources.

FIG. 3 is a perspective view of an add-on device to a hand pump.

FIG. 4A-4C are perspective views of the add-on device according to aspects of the disclosure.

FIG. 5A-5B are cross-sectional views of the add-on device according to aspects of the disclosure.

FIG. 6 is a cross-sectional view of the add-on device according to aspects of the disclosure.

In accordance with common practice, the various features illustrated in the drawings are not drawn to scale. Accordingly, the dimensions of the various features may be arbitrarily expanded or reduced for clarity. In addition, some of the drawings may not depict all of the components of a given system, method or device. Finally, like reference numerals denote like features throughout the specification and figures.

### DETAILED DESCRIPTION

Numerous details are described herein in order to provide a thorough understanding of the example implementations illustrated in the accompanying drawings. However, some implementations may be practiced without many of the specific details, and the scope of the claims is only limited by those features and aspects specifically recited in the claims. Furthermore, well-known processes, components, and materials have not been described in exhaustive detail so as to avoid obscuring pertinent aspects of the implementations described herein.

The disclosure includes an add-on device to provide mechanical leverage for a hand pump is configured to fluidically couple with an IV bag and accelerate a fluid from the IV bag to a patient for treatment. In particular, the add-on device is configured to compress (or squeeze) the hand pump. When a hand pump is compressed, a flow of fluid from the IV bag to a patient is increased. Additionally, the add-on device to the hand pump reduces fatigue experienced by clinicians when compressing hand pumps incorporated in conventional IV sets.

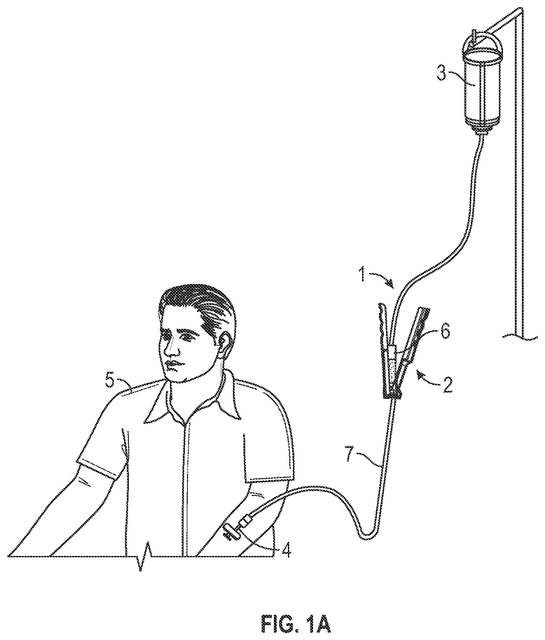

As shown in FIG. 1A, the IV set 1 and IV set hand pump 6 may be coupled to a fluid source 3 and to a catheter 4 inserted into a patient 5. Here, if the maximum fluid flow rate from the infusion pump system is not sufficient, the IV set hand pump 6, may be activated (e.g., squeezed and released rythmically) to force the fluid to flow more quickly. An add-on device 2 may be used to compress (or squeeze) the hand pump more easily.

In aspects of the disclosure, the IV set hand pump 6, may include any suitable fastener to couple the IV set hand pump to an IV pole, a bed, an operating table and the like. For example, the fastener may be a cradle, a hangar, a hook, Velcro®, adhesive, and/or any other suitable fastener. In aspects of the disclosure, the IV set hand pump 6, may be configured to simply hang in line with an IV set (e.g., IV set 1) via IV tubing (e.g., IV tubing 7). Here, the IV set hand pump 6 may be positioned on the IV set to maximize accessibility and/or to keep the IV set hand pump 6 out of specific work areas (e.g., right above patient).

In aspects of the disclosure, the geometry and/or shape of the add-on device may be varied based on hand size and/or number of finger grips. For example, there may be small, medium and large sizes for use by different sized users. In aspects of the disclosure, the shaped portion and the grip members of the grip portion may be sized and shaped in any suitable manner. In aspects of the disclosure, the flow rates may be configured based on size/shape of the body and diameter of the inlet ports and outlet ports.

The IV pump set hand pump 6 and add-on device 2 can be used in many applications. For example, blood transfusions are potential life-saving procedures that can help replace blood lost due to surgery or injury. These procedures can also help if an illness prevents human body from making blood or some of the blood's components correctly. In aspects of the disclosure, drugs may be administered to a patient quickly via a manual hand pump that forces the drugs via the IV line at the rate with which user is pumping.

FIG. 1B illustrates an IV pump administering a medical fluid to a patient. The IV pump 30 includes a controller 32 and one or more pump modules 34. An IV set 20 is connected between a container 36 (e.g., an IV bag) of the medical fluid and the patient 5. During operation, IV pump 30 delivers the medical fluid to the patient 5. The IV pump 30 is configured to administer the medical fluid to the patient at a predetermined and uniform rate. In some implementations, the IV set 20 is utilized to further accelerate a flow of medical fluid from the container 36 (or the IV pump 30) to the patient.

In some embodiments, the IV set 20 can include a plurality of hand pumps 6 that can be used to further accelerate the flow of medical fluid. In addition, in some implementations, the plurality of hand pumps are used to agitate any particulate trapped within one or more filters (of the IV set 20 and/or the IV pump 30), increasing flow through the filter and the IV set 20.

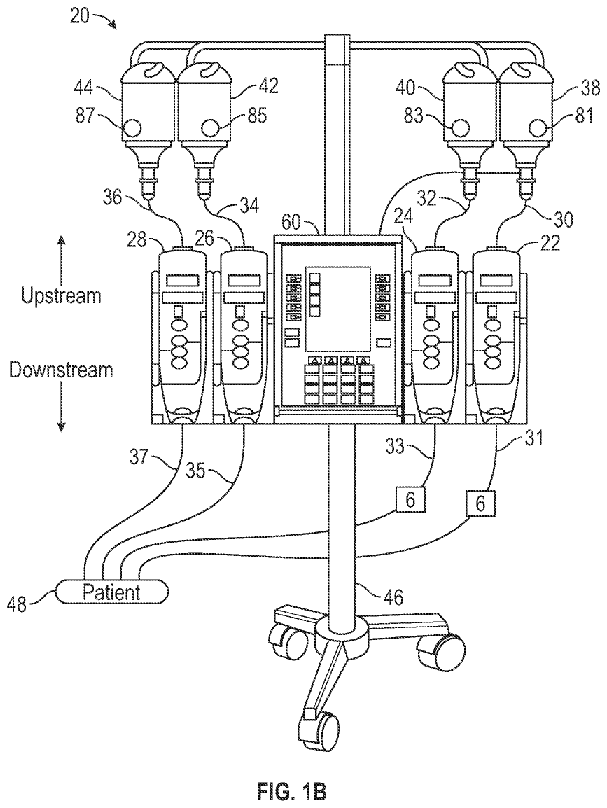

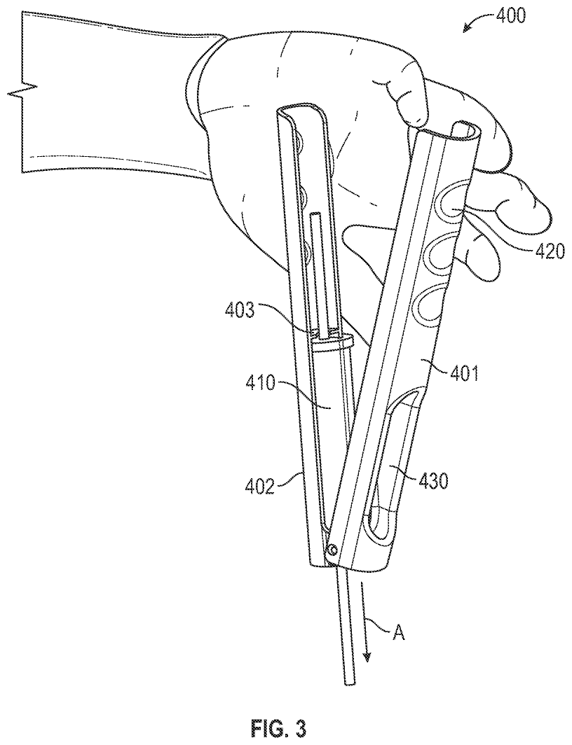

As shown and described below in reference to FIGS. 2-4, the hand pumps 6 may also include an add-on device 2 to increase mechanical leverage when compressing the hand pumps. During operation (i.e., the administration of medical fluid to the patient), the filter(s) can be clogged with particulate, reducing flow through the IV set 20. The filter(s) is configured to prevent the transfer of bacteria, microorganisms, and/or other pathogens. The plurality of hand pumps accelerate the flow of medical fluid and/or agitate any particulate trapped within one or more filters via the application of a force to the plurality of hand pumps (e.g., squeezing the plurality of hand pumps) as discussed below in reference to FIGS. 2-4.

FIG. 2 illustrates an IV set 200. The IV set 200 includes one or more connectors 202, one or more clamps 204 and 208, tubing 206, a drip chamber 210, a hand pump 220, an injection site 230, and an outlet port 232. As described above in reference to FIG. 1B, the IV set 200 delivers fluid from a fluid source, such as a container 48, to a patient 5 through tubing 206. More specifically, fluid from the fluid source is introduced into a first portion of the tubing 206-a of the IV set 200 and delivered to the patient 5 (e.g., via a flow generated by an IV pump and/or a hand pump). The connectors 202 facilitate coupling and/or fluid communication between the first portion of the tubing 206-a and a fluid source (e.g., one or more containers). More specifically, the connectors 202 fluidically couple the first portion of the tubing 206-a to one or more one or more containers. In some implementations, the connectors 202 can be connector spikes that pierce a membrane of the container to permit fluid communication from the container into the tubing 206. Alternatively, in some implementations, the connectors 202 can be needleless connectors to avoid inadvertent piercing of membranes when fluidically coupling the first portion of the tubing 206-a and the fluid source. In some implementations, the needleless connectors can include a no-drip feature to prevent leaks or surface contamination. In some implementations, the needleless connectors can further include a luer lock to prevent accidental discharges. In some implementations, the needleless connectors engage with fluid containers that include mating connectors.

In some implementations, a second portion of the tubing 206-b is coupled to the patient 5 via the outlet port 232. Additional medical fluids or treatments can be introduced to the patient via the IV set 200. In some implementations, additional medical fluids or treatments can be introduced into the IV set 200 via the injection site 230. In some implementations, the fluid container allows for pulling aliquots of blood for analysis.

Clamps 204 and 208 are configured to control fluid flow through the tubing 206 of the IV set 200. For example, clamps 204 and 208 can occlude the flow of fluid through the tubing 206 such that no fluid is distributed to the patient. Alternatively, clamps 204 and 208 can partially occlude the flow of fluid through the tubing 206 such that the flow of fluid distributed to the patient is reduced. The clamps 204 and 208 can be roller clamps, pinch clamps, slide clamps, and/or other clamps known in the art.

In some implementations, the drip chamber 210 is formed of a transparent material or a semi-transparent material to provide a visual indicator of the flow rate of a medical fluid therethrough. The drip chamber 210 is configured to allow clinicians and/or other medical practitioners to monitor and adjust a flow rate of the medical fluid based on the visual indicator provided by the drip chamber 210 (e.g., counting the number of drops per second). During operation, medical fluid can drip or otherwise flow through the chamber volume of the drip chamber 210. Medical fluid can enter the drip chamber 210 through an upper portion or inlet portion 212 defined in the drip chamber 210. The inlet portion 212 is in fluid communication with the first portion of tubing 206-a. Fluid flow exits the drip chamber 210 through a lower portion or outlet portion 214. The outlet portion 214 can be in fluid communication with the second portion of the tubing 206-b. In some implementations, the drip chamber 210 can equalize pressure differentials between the chamber volume and the environment during operation. In some implementations, the drip chamber 210 can be formed from a resilient material to allow the drip chamber 210 to be squeezed or compressed to draw in medical fluid for priming of an IV system. In some implementations, the drip chamber 210 draws in medical fluid for priming the IV system. As can be appreciated, the drip chamber 210 can be filled with a desired volume of medical fluid during the priming operation.

In some implementations, the drip chamber 210 includes a filter for filtering the medical fluid passing therethrough. In some implementations, the filter 213 is disposed within the drip chamber 210. In some implementations, the filter 213 is disposed within the inlet portion 212 or the outlet portion 214 of the drip chamber 210. Alternatively, in some implementations, the filter 213 is disposed within the chamber volume of the drip chamber 210. In some implementations, the filter 213 is an integral part of the drip chamber 210. During operation, fluid can flow through the inlet portion 212 of the drip chamber 210, through a filter 213 to an outlet portion 214 of the drip chamber 210. As medical fluid flows through the drip chamber 210, the fluid can be filtered prior to flowing out of the drip chamber 210 and through the second portion of tubing 206-b. As described above in reference to FIG. 1, fluid within the chamber volume of the drip chamber 210 can pass through the filter 213 to prevent the transfer of bacteria, microorganisms, and/or other pathogens to the patient. As can be appreciated, a positive pressure differential can direct fluid flow through the filter 213. As described herein, the filter 213 can selectively filter the flow through the drip chamber 210. The filter 213 can have an average filter opening ranging between 15 to 200 microns. In some implementations, the average filter opening can range between 180 to 200 microns. Optionally, the filter 213 can have pores that vary in size. In some implementations, the filter 213 can be formed from a non-woven filter material.

During operation, the filter 213 can become clogged with particulate, limiting the filtering efficiency and flow through the filter 213. The conventional IV set 200 can include a hand pump 220 to dislodge sediment or particulate embedded in the filter 213, extending the life of the filter 213 and increasing the flow through the filter 213. The hand pump 220 can direct back flow or back pressure through the filter 213 to dislodge particulate embedded in the filter 213. In the depicted example, a clinician can actuate the hand pump 220 to create back flow through the filter 213. Typically, the hand pump is a typical flexible cylinder that is squeezed by a user continuously with one hand and released before activating or squeezing it again.

IV sets may be formed from any combination of infusion components and tubing. Typically, the infusion components and tubing are disposable products that are used once and then discarded. The infusion components and tubing may be formed from any suitable material (e.g., plastic, silicone, rubber), many or all of which are clear or translucent so that the fluid flow or levels inside can be seen.

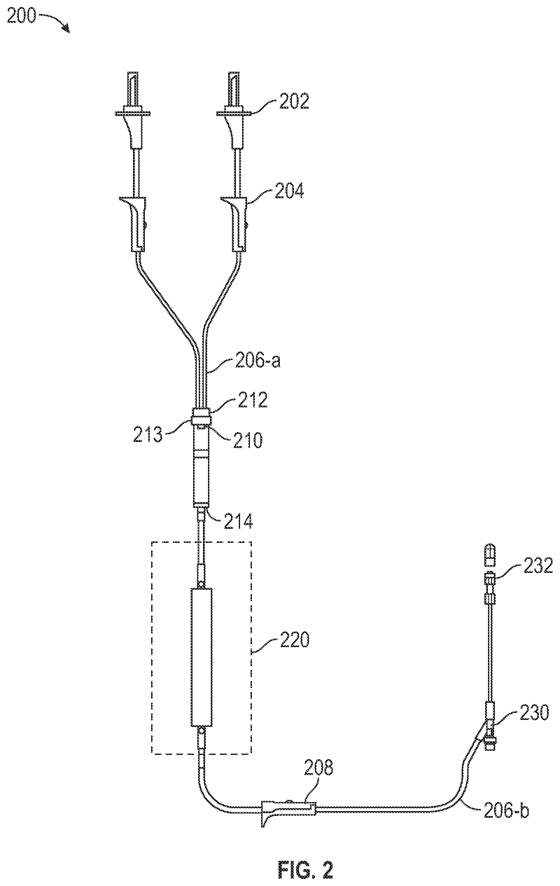

FIGS. 3-6 illustrate the add-on device (also referred to as “device” herein) 400 for a hand pump 410 in accordance with aspects of the present disclosure. The device 400 may be a standalone reusable device for pumping more blood through a hand pump using less force. In some embodiments, the add-on device 400 is a standalone reusable device with a hinged design consisting of two parts, a first member 401 and a second member 402. In some embodiments, the device 400 is placed on the hand pump 410 and the first member 401 of the device is configured to actuate the hand pump 410. In some embodiments, the first member 401 and the second member 402 also have a grip 420 provided to handle the device with ease. In some embodiments, the position of device 400 is not fixed and can be altered based on comfort level of user.

The add-on device 400 is compatible with existing blood sets. Existing hand pumps 410 are placed inside the second member 402 of device 400 and then the first member 401 is hingedly connected to the second member 402. The second member 402 may comprise a ledge 403 configured to secure the hand pump 410 in the device 400. Arrow A, as shown in FIG. 3, illustrates the direction of blood flow through the hand pump 410 in the device 400. The mechanical leverage provided by the device amplifies the force applied to the hand pump 410, which lessens the amount of force that needs to be applied by the user.

In some embodiments, the device 400 is reusable by disconnecting with a first IV set and attaching to a second IV set without disconnecting either the first or second IV sets from a fluid source or from a delivery catheter assembly.

The device 400 receives the hand pump 410 inside the second member 402 and the first member 401 is actuated by hand by a user of the device 400. The first member 401 and/or the second member 402 may have contours to help in gripping during actuation. The opening range of device 400 may be set at the level that provides the best ergonomics for gripping. In some embodiments, the device 400 is designed to provide a mechanical leverage of three. This leverage can be increased but may be set considering the strength of the hand pump 410 material and ergonomics. The position of device 400 is not fixed. The device 400 enables a higher flow rate to be achieved than existing hand pumps on their own.

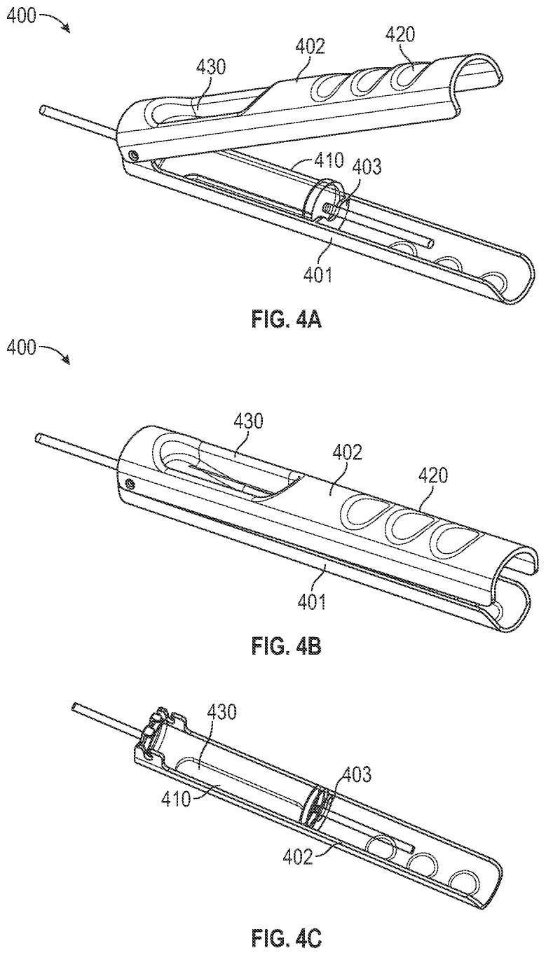

As depicted in FIGS. 5A-5B, the device 400 includes a compression section 415 that is configured to compress the hand pump 410 within the device 400. For example, the compression section 415 can include portion of the first member 401 that extends into the hand pump 410 when the first member 401 is compressed towards the second member 402. As illustrated, the compression section 415 may include a portion of the first member 401 that gradually compresses the hand pump 410 by compressing a portion of the hand pump 410 closest to the rotational axis and continues to compress sequential portions of the hand pump 410 as the first member 401 continues to rotate toward the second member 402. In some embodiments, the compression section 415 can include arcuate portions that have varying degrees of curvature along the first member 401 such that the hand pump 410 is compressed in a rolling fashion from one end of the hand pump 410 toward an opposite end of the hand pump 410 as the first member 401 is rotates toward the second member 402.

In some embodiments, the first member 401 and/or the second member 402 includes an opening or a window 430 that allows the user to view the handpump when compressing the device 400.

In aspects of the disclosure, the grip portion 420 comprises a shaped surface having one or more grip members comprising at least one of a circular shape, an oblong shape and an outer arcuate groove, wherein each grip member is sized and shaped to receive a finger of a hand. In aspects of the disclosure, the grip members are textured, and wherein texture members are disposed on an exterior surface of the body, the texture members comprising one of bumps, raised lines and non-slip coated areas.

In aspects of the disclosure, the one or more grip members are configured to be gripped by one of a robotic arm and mechanical pump fingers. In aspects of the disclosure, the body is configured to be gripped by one of a robotic arm and mechanical pump fingers. In aspects of the disclosure, an exterior surface of the body comprises texture members. In aspects of the disclosure, the texture members are bumps. In aspects of the disclosure, the texture members are raised lines. In aspects of the disclosure, the texture members are non-slip coated areas.

In one or more embodiments, an IV set comprises a first IV tube configured to be coupled to a fluid source, a second IV tube, and an IV set hand pump, and an add-on device comprising a grip portion, wherein the IV set hand pump is configured, when squeezed, to increase a fluid flow rate of the IV set downstream of the IV set hand pump beyond a maximum gravity based fluid flow rate of the IV set.

In aspects of the disclosure, the size and/or shape of the device 400 may be varied based on hand size and/or number of finger grips. For example, there may be small, medium and large sizes for use by different sized users. In aspects of the disclosure, the shaped portion and the grip members of the grip portion may be sized and shaped in any suitable manner. In aspects of the disclosure, the flow rates may be configured based on size/shape of the device and how much mechanical leverage it provides. In aspects of the disclosure, the size and/or shape of the device 400 may be varied based on the size of the hand pump being used.

In some embodiments, the device 400 may be made of any suitable rigid material (for e.g., a plastic or a metal) to provide a desired amount of mechanical advantage and life-cycle usage.

Embodiments of the present disclosure provide a method for using an add-on device for an intravenous (IV) set hand pump comprising coupling a first IV tube of an IV set to a fluid source, coupling a second IV tube of the IV set to a fluid recipient, installing the device to the IV set hand pump coupled between the first IV tube and the second IV tube, compressing the add-on device to force fluid to flow through the IV set hand pump at a flow rate greater than one of a maximum gravity-based fluid flow rate of the IV set and a maximum infusion flow rate of an infusion pump, and repeating the compressing and expanding steps to deliver a desired volume of fluid to the fluid recipient.

In some embodiments, the add-on device comprises a first member configured to actuate the handpump, and a second member hingedly connected to the first member and configured to receive the hand pump, wherein, when actuated, the add-on device is configured to increase a fluid flow rate downstream of the IV set hand pump beyond a maximum gravity-based fluid flow rate of the IV set. In some embodiments, the first member and/or the second member comprises a grip portion. In some embodiments, the grip portion comprises a shaped surface having one or more grip members comprising at least one of a circular shape, an oblong shape and an outer arcuate groove, wherein each grip member is sized and shaped to receive a finger of a hand. In some embodiments, the method further comprises reusing the device by disconnecting with a first IV set and attaching to a second IV set without disconnecting either the first or second IV sets from a fluid source or from a delivery catheter assembly.

Embodiments of the present disclosure provide a method for increasing fluid flow rate downstream of an IV set hand pump beyond a maximum gravity-based fluid flow rate of the IV set comprising coupling a first IV tube of the IV set to a fluid source, coupling a second IV tube of the IV set to a fluid recipient, installing an add-on device to the IV set hand pump coupled between the first IV tube and the second IV tube, compressing the add-on device to force fluid to flow through the IV set hand pump. In some embodiments, the add-on device comprises a first member configured to actuate the handpump; and a second member hingedly connected to the first member and configured to receive the hand pump; wherein, when actuated, the add-on device is configured to increase a fluid flow rate downstream of the IV set hand pump beyond a maximum gravity-based fluid flow rate of the IV set.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

### Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1: An add-on device for an intravenous (IV) set hand pump comprising a first member configured to actuate the handpump; and a second member hingedly connected to the first member and configured to receive the hand pump; wherein, when actuated, the add-on device is configured to increase a fluid flow rate downstream of the IV set hand pump beyond a maximum gravity-based fluid flow rate of the IV set.
Clause 2: The add-on device of clause 1, wherein the first member comprises a compression section that extends into the hand pump when the first member is compressed towards the second member.
Clause 3: The add-on device of clause 2, wherein the compression section includes a portion of the first member that gradually compresses the hand pump by compressing a portion of the hand pump closest to the rotational axis and continues to compress sequential portions of the hand pump as the first member continues to rotate toward the second member.
Clause 4: The add-on device of clause 2, wherein the compression section includes arcuate portions that have varying degrees of curvature along the first member such that the hand pump is compressed in a rolling fashion from one end of the hand pump toward an opposite end of the hand pump as the first member is compressed towards the second member.
Clause 5: The add-on device of clause 1, wherein the second member further comprises a ledge configured to secure the hand pump in the add-on device.
Clause 6: The add-on device of clause 1, wherein the first member and/or the second member comprises a grip portion.
Clause 7: The add-on device of clause 6, wherein the grip portion comprises a shaped surface having one or more grip members comprising at least one of a circular shape, an oblong shape and an outer arcuate groove.
Clause 8: The add-on device of clause 6, wherein each grip member is sized and shaped to receive a finger of a hand.
Clause 9: The add-on device of clause 1, wherein the distance between the first member and the second member is not fixed.
Clause 10: The add-on device of clause 1, wherein the device is reusable by disconnecting with a first IV set and attaching to a second IV set without disconnecting either the first or second IV sets from a fluid source or from a delivery catheter assembly.
Clause 11: The add-on device of clause 1, wherein the device provides a mechanical leverage of three.
Clause 12: The add-on device of clause 1, wherein the first member and/or the second member further comprises a window for viewing the hand pump.
Clause 13: The add-on device of clause 1, wherein the overall length of the add on device is 225 mm, 230 mm, 235, mm, 240 mm, 245 mm, 250 mm, 255 mm, 260 mm, 265 mm, or 270 mm.
Clause 14: A method for using an add-on device for an intravenous (IV) set hand pump comprising coupling a first IV tube of an IV set to a fluid source; coupling a second IV tube of the IV set to a fluid recipient; installing the device to the IV set hand pump coupled between the first IV tube and the second IV tube; compressing the add-on device to force fluid to flow through the IV set hand pump at a flow rate greater than one of a maximum gravity-based fluid flow rate of the IV set and a maximum infusion flow rate of an infusion pump; and repeating the compressing and expanding steps to deliver a desired volume of fluid to the fluid recipient.
Clause 15: The method of clause 14, wherein the add-on device comprises a first member configured to actuate the handpump; and a second member hingedly connected to the first member and configured to receive the hand pump; wherein, when actuated, the add-on device is configured to increase a fluid flow rate downstream of the IV set hand pump beyond a maximum gravity-based fluid flow rate of the IV set.
Clause 16: The method of clause 14, wherein the first member and/or the second member comprises a grip portion.
Clause 17: The method of clause 17, wherein the grip portion comprises a shaped surface having one or more grip members comprising at least one of a circular shape, an oblong shape and an outer arcuate groove, wherein each grip member is sized and shaped to receive a finger of a hand.
Clause 18: The method of clause 14 further comprising reusing the device by disconnecting with a first IV set and attaching to a second IV set without disconnecting either the first or second IV sets from a fluid source or from a delivery catheter assembly.
Clause 19: A method for increasing fluid flow rate downstream of an IV set hand pump beyond a maximum gravity-based fluid flow rate of the IV set comprising coupling a first IV tube of the IV set to a fluid source; coupling a second IV tube of the IV set to a fluid recipient; installing an add-on device to the IV set hand pump coupled between the first IV tube and the second IV tube; and compressing the add-on device to force fluid to flow through the IV set hand pump.
Clause 20: The method of clause 19, wherein the add-on device comprises a first member configured to actuate the handpump; and a second member hingedly connected to the first member and configured to receive the hand pump; wherein, when actuated, the add-on device is configured to increase a fluid flow rate downstream of the IV set hand pump beyond a maximum gravity-based fluid flow rate of the IV set.

### FURTHER CONSIDERATION

In some implementations, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

It will be understood that, although the terms “first,” “second,” etc. may be used herein to describe various elements, these elements should not be limited by these terms. These terms are only used to distinguish one element from another. Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

The terminology used herein is for the purpose of describing particular implementations only and is not intended to be limiting of the claims. As used in the description of the implementations and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such an implementation may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various implementations for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

### EXAMPLES

### Example 1: Add-On Device Mechanical Advantage

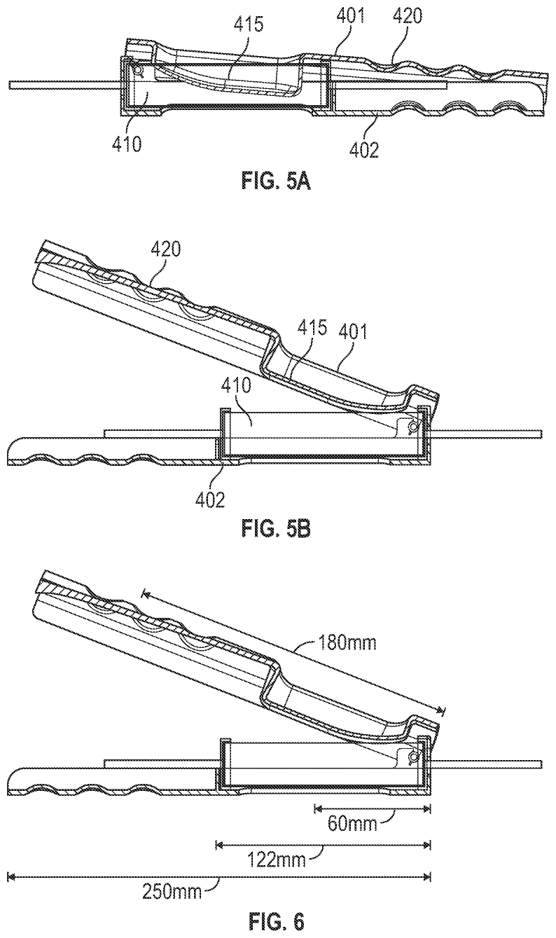

TABLE 1
Mechanical Advantage of an Embodiment
of an Add-On Device (FIG. 6)

| | |
|---|---|
| Length of the Device | 230 mm |
| Length of Hand Pump | 122 mm |
| Distance from Hinge point to center | 60 mm |
| of Hand pump | |
| Distance from Hinge point to center | 180 mm |
| of Grip | |
| Mechanical Advantage | 180 mm/60 mm = 3 |

The Activation Force is 6 lbf in one embodiment of a hand pump which is reduced to 2 lbf after installing the add-on device. With the same force of 6 lbf, the user can triple the amount of blood pumped through the hand pump.
With this add-on device, the user pumps the blood for more time or he/she squeezes the pump more frequently reducing the overall pumping time depending upon his/her comfort level. Due to reduced effort, the user can squeeze the hand pump three times faster, which will increase fluid displaced per hour by three times.

TABLE 2
Flow Rate & Pumping Time Calculations
Time to displace 8.2 Liter of Fluid with hand pump alone - 1 hour

| | | Total Head | Vol. per stroke | Delivery Time (s) | | | | | Activation |
|---|---|---|---|---|---|---|---|---|---|
| | liters/hr. | (M of Blood) | avg. ml \| std deviation | Total | Pump | Refill | Std Dev | Shape | force (lbf) |
| BD | 8.200 | 11.2910 | 19.5 \| 3.2 | 7.0 | 2.5 | 4.5 | 1.7 | Cylinder | 6.0 |
| Baxter | 15.200 | 11.2916 | 20.8 \| 3.7 | 5.0 | 2.2 | 2.8 | 0.9 | Cylinder | 4.0 |
| B Braun | 13.100 | 11.2925 | 21.1 \| 2.2 | 5.8 | 2.2 | 3.6 | 0.8 | Cylinder | 3.2 |
| ICU Medical | 13.600 | 11.2924 | 21.5 \| 3.9 | 5.8 | 2.3 | 3.4 | 0.8 | Bulb | 10.3 |
| CODAN | 9.400 | 11.2923 | 23.4 \| 2.0 | 9.1 | 3.8 | 5.3 | 1.2 | Cylinder | 4.1 |

Time to displace 8.2 Liter of Fluid with add-on device—approximately 20 minutes

What is claimed is:

1. An add-on device for an intravenous (IV) set hand pump comprising:
a first member configured to actuate the hand pump; and
a second member hingedly connected to the first member and configured to receive the hand pump;
wherein, when actuated, the add-on device is configured to increase a fluid flow rate downstream of the IV set hand pump beyond a maximum gravity-based fluid flow rate of the IV set.

2. The add-on device of claim 1, wherein the first member comprises a compression section that extends into the hand pump when the first member is compressed towards the second member.

3. The add-on device of claim 2, wherein the compression section includes a portion of the first member that gradually compresses the hand pump by compressing a portion of the hand pump closest to a rotational axis of the add-on device and continues to compress sequential portions of the hand pump as the first member continues to rotate toward the second member.

4. The add-on device of claim 2, wherein the compression section includes arcuate portions that have varying degrees of curvature along the first member such that the hand pump is compressed in a rolling fashion from one end of the hand pump toward an opposite end of the hand pump as the first member is compressed towards the second member.

5. The add-on device of claim 1, wherein the second member further comprises a ledge configured to secure the hand pump in the add-on device.

6. The add-on device of claim 1, wherein the first member and/or the second member comprises a grip portion.

7. The add-on device of claim 6, wherein the grip portion comprises a shaped surface having one or more grip members comprising at least one of a circular shape, an oblong shape and an outer arcuate groove.

8. The add-on device of claim 6, wherein each grip member is sized and shaped to receive a finger of a hand.

9. The add-on device of claim 1, wherein a distance between the first member and the second member is not fixed.

10. The add-on device of claim 1, wherein the device is reusable by disconnecting with a first IV set and attaching to a second IV set without disconnecting either the first or second IV sets from a fluid source or from a delivery catheter assembly.

11. The add-on device of claim 1, wherein the device provides a mechanical leverage of three.

12. The add-on device of claim 1, wherein the first member and/or the second member further comprises a window for viewing the hand pump.

13. The add-on device of claim 1, wherein an overall length of the add on device is 225 mm, 230 mm, 235, mm, 240 mm, 245 mm, 250 mm, 255 mm, 260 mm, 265 mm, or 270 mm.

14. A method for using an add-on device for an intravenous (IV) set hand pump comprising:
coupling a first IV tube of an IV set to a fluid source;
coupling a second IV tube of the IV set to a fluid recipient;
installing the device to the IV set hand pump coupled between the first IV tube and the second IV tube;
compressing the add-on device to force fluid to flow through the IV set hand pump at a flow rate greater than one of a maximum gravity-based fluid flow rate of the IV set and a maximum infusion flow rate of an infusion pump; and
repeating the compressing and expanding steps to deliver a desired volume of fluid to the fluid recipient.

15. The method of claim 14, wherein the add-on device comprises:
a first member configured to actuate the hand pump; and
a second member hingedly connected to the first member and configured to receive the hand pump;
wherein, when actuated, the add-on device is configured to increase a fluid flow rate downstream of the IV set hand pump beyond a maximum gravity-based fluid flow rate of the IV set.

16. The method of claim 15, wherein the first member and/or the second member comprises a grip portion.

17. The method of claim 16, wherein the grip portion comprises a shaped surface having one or more grip members comprising at least one of a circular shape, an oblong shape and an outer arcuate groove, wherein each grip member is sized and shaped to receive a finger of a hand.

18. The method of claim 14 further comprising reusing the device by disconnecting with a first IV set and attaching to a second IV set without disconnecting either the first or second IV sets from a fluid source or from a delivery catheter assembly.

19. A method for increasing fluid flow rate downstream of an IV set hand pump beyond a maximum gravity-based fluid flow rate of the IV set comprising:
coupling a first IV tube of the IV set to a fluid source;
coupling a second IV tube of the IV set to a fluid recipient;
installing an add-on device to the IV set hand pump coupled between the first IV tube and the second IV tube; and
compressing the add-on device to force fluid to flow through the IV set hand pump.

20. The method of claim 19, wherein the add-on device comprises:
a first member configured to actuate the hand pump; and
a second member hingedly connected to the first member and configured to receive the hand pump;
wherein, when actuated, the add-on device is configured to increase a fluid flow rate downstream of the IV set hand pump beyond a maximum gravity-based fluid flow rate of the IV set.
